(19) United States
(12) Patent Application Publication
(10) Pub. No.: US 2006/0018829 A1
(43) Pub. Date: Jan. 26, 2006

(54) Cellular receptors utilized as carrier agents for pharmaceutical compounds used in tumor imaging and cancer treatment

(76) Inventors: Henry John Smith, San Jacinto, CA (US); James Roger Smith, Aliso Viejo, CA (US)

Correspondence Address: STETINA BRUNDA GARRED & BRUCKER, 75 ENTERPRISE, SUITE 250, ALISO VIEJO, CA, 92656, US

(21) Appl. No.: 10/896,133

(22) Filed: Jul. 22, 2004

(51) Int. Cl. A61K 51/00; A61K 49/04

(52) U.S. Cl. 424/1.49; 424/9.411

(57) ABSTRACT

A method whereby non-immunogenic intraspecies proteins are used as carrier compounds to deliver imaging agents and pharmaceutical drugs to tumors in the human patient. This invention describes the propensity of certain solubilized cellular receptor proteins to localize in necrotic or inflamed areas of tumors but not in healthy normal tissues. Two examples of these receptors are tumor necrosis factor receptor (TNF-R) and the interleukin receptors (IL-R). By combining various pharmaceutical agents with these receptor proteins it is possible to localize these agents within the necrotic or damaged areas of the tumor where they will have the greatest therapeutic effect.

### BACKGROUND OF THE INVENTION

The main applications of this invention are in developing improved methods for Cancer Imaging and Cancer Treatment. One out of every four people in the U.S. will die from cancer. There is tremendous interest in developing improved methods of cancer detection and therapy because the earlier the cancer is detected and treated the better the chances of success. Early research on targeting tumors used antibodies obtained from immunized animals. Subsequent studies have been almost exclusively devoted to developing monoclonal antibodies against tumors.

Much of the research has utilized monoclonal antibodies produced by murine hybridomas. There is however a problem when murine monoclonal antibodies are injected into cancer patients. There is a risk that the patient may develop an immune response against the “foreign” protein making further treatment ineffective. In order to avoid this problem there is intensive research into developing methods to “humanize” the monoclonal antibodies by substituting parts of the mouse antibody with human components or by developing fully human monoclonal antibodies.

This invention describes a new method of targeting tumors using “solubilized cellular receptors” derived from certain types of cells.

Cells communicate with each other using a variety of signaling mechanisms such as hormones, growth factors, cytokines etc. These bind to specific cellular receptors on the surface of the target cell and cause it to respond in a particular fashion. For example, epidermal growth factor will bind to the epidermal growth factor receptor present on epidermal cells, estrogen will bind to estrogen receptors on breast cells, and cytokines will bind to cytokine receptors on inflammatory cells. Normally, the signaling ligand is soluble and travels to its receptor site on the target cell. The targeted cellular receptor is immobile as it is incorporated as part of the target cell's membrane. The novelty of this invention is in its description of the reverse process—the use of “solubilized” cellular receptors to target the location of the signaling ligand wherever it is concentrated.

There are certain types of cellular receptors that have the ability to bind to substances present in tumors and/or areas of inflammation. This is exemplified by the type of receptors known as tumor necrosis factor receptors (TNF-R) and by the interleukin receptors (IL-R). For illustrative purposes the use of Tumor Necrosis Factor Receptor (TNF-R) is described here. However, the use of other cellular receptors such as the interleukin receptors as exemplified by the interleukin 2 receptor (IL2-R) receptor and the interleukin 6 receptor (IL6-R) can be employed in like manner and are considered within the scope of this invention.

Tumor necrosis factor (TNF) is a cytokine that can bind to other immune cells and stimulate them to participate in the immune reaction. These cells have specific receptors on their surface called tumor necrosis factor receptors (TNF-R). TNF-R can be prepared and solubilized and used as a “carer” protein to deliver pharmacological compounds to the tumor. Many tumors have areas of necrosis which have elevated levels of tumor necrosis factor and therefore solubilized TNF-R will bind to the TNF present in these areas and become localized within the tumor.

By combining various cancer imaging or anti-cancer drugs with solubilized TNF-R it is possible to transport the pharmaceutical compound within the necrotic areas of tumors. The pharmaceutical agent will then have a cytotoxic effect upon the surrounding tumor tissue. Normal healthy tissues have little or no TNF and there will be little binding of the labeled carrier protein within normal tissue and less exposure to the cytotoxic agent.

A further benefit of this invention is that because the cellular receptors are derived from human cells they are non-antigenic to the cancer patient, and can therefore be used repeatedly as “carriers” for cancer imaging and cancer therapy compounds without provoking an immune response in the patient.

### SUMMARY OF THE INVENTION

This invention describes the novel use of non-immunogenic intraspecies proteins as carrier agents for pharmaceutical compounds used to diagnose and treat various diseases. In contrast to conventional methods which seek to produce anti-tumor antibodies this invention identifies a different class of binding proteins known as cellular receptors which can bind to “naturally” occurring compounds such as hormones or growth factors or cytokines etc.

Many tumors have necrotic areas containing elevated levels of a substance called tumor necrosis factor (TNF). Tumor necrosis factor is a cytokine which can stimulate other immune cells by binding to specific receptors on the cell. These receptors are called tumor necrosis factor receptors (TNF-R). It is possible to isolate or produce solubilized TNF-R and to use these as carrier proteins by combining them with various cancer imaging and anti-cancer drugs. When injected into the cancer patient the labeled TNF-R carrier protein will bind to and localize within the necrotic areas found in many tumors. The anti-cancer agents will then have an effect upon the surrounding tumor tissue. As normal healthy tissues have little or no tumor necrosis factor present the labeled carrier proteins cannot bind to normal tissue and there will be less cytotoxic effect upon normal tissue.

The TNF-R carrier proteins described here are non-immunogenic, and therefore can be used repeatedly over a prolonged period of time to diagnose and treat tumors.

### DESCRIPTION OF THE INVENTION

This invention describes a method for improved delivery of diagnostic and pharmaceutical agents to tumors. It describes the use of a new type of binding protein called cellular receptors that have the propensity to localize within tumors. These receptors can be used as “carrier” proteins by combining them with various cancer imaging and anti-cancer compounds. The labeled carrier proteins will carry the cancer imaging or cytotoxic anti-cancer agent to the tumor while sparing normal tissues.

This invention describes the use of cellular receptors as exemplified by tumor necrosis factor receptor (TNF-R) as a carrier protein for pharmaceutical drugs. Many tumors contain areas of necrosis and inflammation and these areas also have elevated levels of a cytokine called tumor necrosis factor (TNF) which appears to be involved in the inflammatory response. Certain immune cells appear to have receptors on their surface which can bind to the tumor necrosis factor and these receptors are designated as tumor necrosis factor receptors (TNF-R).

Tumor necrosis factor receptors (TNF-R) can be prepared in several ways. The first method is to extract them from immune cells according to known procedures. Briefly this would involve mechanical disruption of the cells and then isolation and purification of the receptors by conventional laboratory techniques such as ion exchange, gel permeation and reverse-phase chromatography. These procedures are known to those skilled in the art and are considered within the scope of the invention.

Another method is genetic engineering. The genetic makeup of TNF-R is known and TNF-R can be prepared according to conventional genetic engineering methods. These procedures are known to those skilled in the art and are considered within the scope of the invention. For example, the genetic code for TNF-R is cloned using the polymerase chain reaction and attached to plasmid DNA. The altered plasmid DNA is used to transform E. Coli bacteria which are grown in fermentation tanks. The transformed bacteria produce human TNF-R which is purified using standard methods such as ion exchange, gel permeation and reverse-phase chromatography. Alternatively, the recombinant TNF-R can be produced using other recombinant protein expression systems such as Spodoptera frugiperda insect cells without affecting the novelty of this invention. The recombinant TNF-R may be expressed either complete, or as a fragment which has TNF binding capacity, or as a fusion protein, without affecting the novelty of this invention. In this context, TNF-R refers to either the complete TNF-R receptor, or the binding fragment of TNF-R, or TNF-R as a component of a fusion protein molecule.

For illustrative purposes the use of Tumor Necrosis Factor Receptor (TNF-R) is described here. However, the use of other cellular receptors such as the interleukin receptors as exemplified by the interleukin 2 receptor (IL2-R) receptor and the interleukin 6 receptor (IL-6R) can be employed in like manner and are considered within the scope of this invention.

Tumor Imaging

For tumor imaging studies there are a variety of radionuclides including Tc-99m, I-123, I-125, In-111, In-113m, Ga-67, or other gamma-emitters. The carrier protein can be iodinated using the chloramine-T method to label the protein with I-125 or 1-131. Other radionuclides may be attached to the carrier TNF-R by chelation with benzyl EDTA or DPTA conjugation procedures. These procedures are known to those skilled in the art and are considered within the scope of this invention.

The radionuclide labeled carrier TNF-R is then injected into the cancer patient where it comes into contact with the tumor tissue. Many tumors contain areas of necrosis with high levels of TNF. The labeled TNF-R will bind to the TNF and the radioactivity will become localized within the necrotic areas of the tumor. In contrast, normal tissues contain healthy intact cells and no free TNF, so the TNF-R will not bind to healthy tissue. The quantity of radioactivity in different tissue locations is measured using gamma ray scanning or tissue sampling techniques. As even small tumors contain areas of necrosis this method may be useful in detecting early tumors.

Another method of tumor detection using this invention is to combine the carrier TNF-R with a radiopaque compound such as barium compounds, gallium compounds, and thallium compounds. The methods of combining proteins to these compounds are known to those skilled in the art and are considered within the scope of this invention. When injected into the cancer patient the radiopaque labeled TNF-R will localize within the necrotic areas of the tumor and is detected by X-radiography.

Another method of tumor detection employs magnetic resonance technology using magnetic resonance-enhancing compounds such as gadolinium, copper, iron, and chromium. The methods of combining protein to these compounds are known to those skilled in the art and are considered within the scope of this invention. When injected into the cancer patient the TNF-R labeled with the magnetic resonance-enhancing compounds will localize within the necrotic areas of the tumor and is detected by magnetic resonance imaging equipment.

Cancer Treatment

There are a wide variety of antineoplastic agents known. These can be classified into the following groups.

The radiologic group includes alpha-emitting and beta-emitting radionuclides such as I-131, Yt-99, Cu-67, Au-198, P-32, and other cytotoxic radionuclides. The radionuclides can be conjugated to the carrier TNF-R using methods that are familiar to those skilled in the art. For example, The carrier protein can be iodinated using the chloramine-T method to label the protein with I-125 or. 1-131. Other radionuclides may be attached to the carrier TNF-R by chelation with benzyl EDTA or DPTA conjugation procedures. For cancer treatment a high dosage of radioactivity is employed. The labeled carrier protein is then injected into the cancer patient where it will localize in the necrotic regions within the tumor. From there the radiation will penetrate into the surrounding tumor where it will have a cytotoxic effect upon the tumor cells.

The cytotoxic drug group includes the folate inhibitors, pyrimidine analogs, purine analogs, alkylating agents and antibiotics. Specific examples include acivicin, aclarubicin, acodazole, adriamycin, ametantrone, aminoglutethimide, anthramycin, asparaginase, azacitidine, azetepa, bisantrene, bleomycin, busulfan, cactinomycin, calusterone, caracemide, carboplatin, carmustine, carubicin, chlorambucil, cisplatin, cyclophosphamide, cytarabine, dacarbazine, dactinomycin, daunorubicin, dezaguanine, diaziquone, doxorubicin, epipropidine, etoposide, etoprine, floxuridine, fludarabine, fluorouracil, fluorocitabine, hydroxyurea, iproplatin, leuprolide acetate, lomustine, mechlorethamine, megestrol acetate, melengestrol acetate, mercaptopurine, methotrexate, metoprine, mitocromin, mitogillin, mitomycin, mitosper, mitoxantrone, mycophenolic acid, nocodazole, nogalamycin, oxisuran, peliomycin, pentamustine, porfiromycin, prednimustine, procarbazine hydrochloride, puromycin, pyrazofurin, riboprine, semustine, sparsomycin, spirogermanium, spiromustine, spiroplatin, streptozocin, talisomycin, tegafur, teniposide, teroxirone, thiamiprine, thioguanine, tiazofurin, triciribine phosphate, triethylenemelamine, trimetrexate, uracil mustard, uredepa, vinblastine, vincristine, vindesine, vinepidine, vinrosidine, vinzolidine, zinostatin and zorubicin. Also included are the toxins such as ricin and diptheria toxin. All these compounds can be conjugated to the carrier TNF-R using methods that are familiar to those skilled in the art. For example, many carboxylic acid-containing compounds such as methotrexate can be conjugated to protein through an active ester intermediate by reacting the compound with N-hydroxysuccinimide and dicyclohexylcarbodiimide; amino sugar containing drugs such as adriamycin and daunomycin may be covalently bound to protein by periodate oxidation of the drug, followed by linking of the oxidized drug to the protein and subsequent reduction of the product with sodium borohydride. The methods of conjugating any particular drug to the carrier protein will vary depending upon the nature of the drug. However, these are according to conventional laboratory methods and are considered to be within the scope of this invention.

The labeled carrier protein is then injected into the cancer patient where it will localize in the necrotic regions within the tumor. From there the drug will diffuse into the surrounding tissues where it will have a cytotoxic effect upon the tumor cells.

The biological response modifier group includes cytokines such as interferons, angiostatin and immune stimulators such as animal or microbial proteins. These compounds can be conjugated to the carrier TNF-R using methods that are familiar to those skilled in the art. For example, glutaraldehyde may be used to cross-link the free amino groups of the TNF-R and modifier protein. Other methods may be employed using conventional laboratory procedures and are considered to be within the scope of this invention.

The labeled carrier protein is then injected into the cancer patient where it will localize in the necrotic regions within the tumor and have the maximum effect upon the surrounding tissue. The effect may be to stimulate an inflammatory response, or to inhibit the growth of new blood vessels to the tumor as in the case of angiostatin, or to stimulate an immune response within the tumor by the foreign animal or microbial protein.

Non-Immunogenicity of the Carrier Protein

As the carrier cellular receptors such as TNF-R and IL-R are obtained from a human source they are non-immunogenic to the cancer patient. They can therefore be used repeatedly for tumor imaging and for cancer treatment over a prolonged period of time without provoking an immune response from the patient.

What is claimed is:

1. A process of utilizing solubilized human cellular receptors as carrier agents for diagnostic and therapeutic pharmaceuticals used in the diagnosis and treatment of cancer.

2. A process according to claim 1, whereby the solubilized cellular receptor is tumor necrosis factor receptor (TNF-R) either as the complete receptor, or the binding portion thereof, or as part of a fusion protein.

3. A process according to claim 1, whereby the solubilized cellular receptor are interleukin receptors such as IL2-R or IL6-R either as the complete receptor, or the binding portion thereof, or as part of a fusion protein.

4. A process according to claims 1-3 whereby the solubilized cellular receptor will bind to their respective ligands found at elevated levels in areas of necrosis and/or inflammation within tumors.

5. A process of tumor imaging according to claims 1-4 utilizing a variety of radionuclides linked to a carrier receptor which is injected into the cancer patient and followed by gamma ray scanning.

6. A process of tumor imaging according to claims 1-4, utilizing a variety of radiopaque compound linked to a carrier receptor which is injected into the cancer patient and followed by X radiography.

7. A process of tumor imaging according to claims 1-4, utilizing a variety of magnetic resonance enhancing compounds linked to a carrier receptor which is injected into the cancer patient and followed by magnetic resonance measuring equipment.

8. A process of cancer treatment according to claims 1-4, utilizing a therapeutic dosage of a variety of radionuclides linked to a carrier receptor and injected into the cancer patient.

9. A process of cancer treatment according to claims 1-4, utilizing a variety of cytotoxic anti-cancer drugs linked to a carrier receptor and injected into the cancer patient.

10. A process of cancer treatment according to claims 1-4, utilizing a variety of biological response modifiers linked to a carrier receptor and injected into the cancer patient.

11. A process of cancer treatment according to claims 1-4, utilizing a variety of toxins linked to a carrier receptor and injected into the cancer patient.

12. A process of cancer treatment according to claims 1-4, utilizing a variety of foreign animal or microbial protein linked to a carrier receptor and injected into the cancer patient.

13. A process of cancer treatment according to claims 1-4, utilizing a variety of blood vessel growth inhibiting compounds linked to a carrier receptor and injected into the cancer patient.

14. A process according to claims 1-13, whereby the use of non-immunogenic human cellular receptors as carrier agents for cancer diagnostic and cancer treatment compounds can be repeated for a prolonged period of time without eliciting a host immune response in the cancer patient.
